(19) United States
(12) Patent Application Publication
(10) Pub. No.: US 2023/0002705 A1
(43) Pub. Date: Jan. 5, 2023

(54) SANITIZING AND DISINFECTING SUBSTRATES LOADED WITH HYPOCHLOROUS ACID SOLUTION

(71) Applicants: Marion E. Paris, JR., Lincolnton, NC (US); Dianne B. Ellis, Cary, NC (US)

(72) Inventors: Marion E. Paris, JR., Lincolnton, NC (US); Dianne B. Ellis, Cary, NC (US)

(21) Appl. No.: 17/721,867

(22) Filed: Apr. 15, 2022

Related U.S. Application Data
(60) Provisional application No. 63/217,738, filed on Jul. 1, 2021.

(51) Int. Cl. C11D 3/48; A61L 2/18; C11D 17/04; C11D 3/395

(52) U.S. Cl. CPC C11D 3/485; A61L 2/18; C11D 17/049; C11D 3/3953; C11D 3/3956; A61L 2202/25; A61L 2101/06

(57) ABSTRACT

Sanitizing and disinfecting substrates of a nonwoven fabric material are loaded with an HOCl solution having a FAC concentration from about 25 ppm to about 750 ppm and a potential of hydrogen (pH) from about 4.5 pH to about 7.5 pH. The substrate may be a synthetic nonwoven fabric laminate having outer layers of one or more Spunbond synthetic fibers and an inner layer of polyester (PET) or pulp. Alternatively, the substrate may be an organic nonwoven fabric formed from one or more layers of cellulosic fibers. The layers may be bonded together by adhesive bonding, chemical bonding or thermal bonding. The substrates may be disposed within a container for storing and subsequently dispensing the substrates. The substrates may be pre-moistened and disposed within the container, or alternatively, disposed within the container and subsequently moistened with the HOCl solution.

### FIELD OF THE INVENTION

The present invention relates generally to sanitizing and disinfecting substrates loaded with a sanitizing and disinfecting solution. In advantageous embodiments, the invention is a substrate in the form of a nonwoven fabric material loaded with a hypochlorous acid (HOCl) solution for sanitizing and disinfecting a hard surface.

### BACKGROUND OF THE INVENTION

Many facilities, including hospitals, nursing homes, prisons and schools, are highly susceptible to multi-drug resistant organisms (MDROs), commonly referred to as infectious bacteria and viruses. For example, the Centers for Disease Control and Prevention (CDCP) estimates that infections acquired from healthcare and food service facilities kill more individuals each year than vehicle accidents, breast cancer or AIDS. Consequently, the Environmental Protection Agency (EPA) and the Food and Drug Administration (FDA) prescribe effective cleaning, sanitizing and disinfecting products and procedures to be used in facilities that provide healthcare services and/or food services. In response, hospitals, nursing homes, prisons and schools have instituted detailed cleaning, sanitizing and disinfecting protocols along with intensive training programs for environmental services personnel to ensure that areas accessed by patients, staff and the public are clean, hygienic and free of infectious bacteria and viruses.

The aforementioned facilities, especially healthcare facilities, have historically utilized a variety of high, medium and low level cleaning, sanitizing and disinfecting solutions, including formaldehyde, hydrogen peroxide, peracetic acid, chlorine-releasing agents (CRAs), such as sodium hypochlorite, chlorine dioxide, etc., iodophores, phenols and bis-phelols solutions. More recently, solutions of hypochlorous acid (HOCl) have been introduced as an efficacious and environmentally friendly alternative to traditional sanitizers and disinfectants. HOCl is a weak acid formed when chlorine dissolves in water and partially dissociates. As a result, HOCl acts as an oxidizer and a primary sanitizing and disinfecting agent in a chlorine solution. The beneficial characteristics attributed to HOCl include that it is a highly effective mechanism for destroying infectious bacteria and viruses, most notably C. diff, E. coli, MRSA (Staph), Salmonella, Tuberculosis, Human Immunodeficiency Virus (HIV), and Severe Acute Respiratory Syndrome (SARS). Despite being highly effective, HOCl is relatively harmless to humans at concentrations suitable for sanitizing and disinfecting. Thus, HOCl solutions are approved for use as sanitizing and disinfecting agents in hospitals, nursing homes, prisons and schools (collectively referred to herein as “institutional facilities”).

Other sanitizing and disinfecting agents commonly used in institutional facilities are not as environmentally friendly and efficacious as HOCl in destroying harmful bacteria and viruses. As a result, it is not uncommon for individuals to contract serious illnesses from the bacteria and viruses at institutional facilities that are treated with other sanitizers and disinfectants. The inability of other sanitizers and disinfectants to destroy infectious organisms increases healthcare costs and causes physical harm to individuals that would have been prevented with the use of a more effective HOCl sanitizing and disinfecting solution. The recent COVID 19 pandemic has increased awareness for cleaning, sanitizing and disinfecting hard surfaces in commercial businesses, public areas and private spaces in addition to the institutional facilities that are already utilizing highly effective HOCl solutions to combat harmful bacteria and viruses.

Despite being highly effective, HOCl has a limited lifespan of effectiveness, commercially referred to as “shelf life.” Over time HOCl decomposes to chloric acid, hydrochloric acid, and oxygen; none of which separately exhibits the same desirable sanitizing and disinfecting properties as a full strength HOCl solution. The shelf life for a HOCl solution as a sanitizing and disinfecting agent is limited from the time it is produced based on its free available chlorine (FAC) concentration measured in parts per million (ppm). As used herein, the term “free available chlorine” or “FAC” is intended to mean the portion of total chlorine in the solution that is present as HOCl or hypochlorite ion (OCl—). Consequently, it is imperative to take steps to ensure that an effective HOCl solution is being used in a recommended cleaning, sanitizing and disinfecting protocol at institutional facilities, commercial businesses, public areas and private spaces. Specifically, it is essential that the HOCl solution is used within its shelf life acceptable for effective cleaning, sanitizing or disinfecting.

Unfortunately, due to its limited shelf-life, HOCl is not presently commercially available in a convenient and stable form for effective sanitizing and disinfecting. HOCl solution is typically generated in bulk by electrolysis using a relatively expensive and time consuming Electrochemically Activated Water (EAW) process. A portion of the HOCl solution is then transferred into a smaller container for sanitizing and disinfecting, such as a spray cart, spray bottle or liquid dispenser. The production date of the HOCl solution is noted on the smaller container to confirm that the HOCl solution is within its effective shelf life. The HOCl solution should be applied directly onto a hard surface and allowed to dry for a predetermined dwell time for effective sanitizing and disinfecting. Oftentimes, the HOCl solution is sprayed or poured onto the hard surface and then wiped with a cloth or towel to apply the solution evenly over the hard surface. It would be more convenient and less labor intensive if the cloth or towel were pre-moistened with the HOCl solution—similar to a conventional alcohol wipe. However, until now there has been no substrate material known to be suitable for providing a HOCl solution in a more convenient form, and particularly, in the form of a pre-moistened cloth, towel or wipe loaded with a stable HOCl solution.

It is likely that no suitable substrate material has been found to date because the HOCl solution does not adequately wet most synthetic substrates, such as a plastic material made from polymers such as polyester (PET), polyethylene (PE), polypropylene (PP), polyvinylchloride (PVC), nylon, etc. Consequently, the cloth, towel, wipe or the like is not an acceptable carrier and applicator for the HOCl solution. In addition, the HOCl solution tends to attack and denigrate most organic substrates and/or any pulp, filler, binder, adhesive or similar agent applied to the synthetic or organic substrate material. Furthermore, a cloth, towel, wipe or the like (collectively referred to herein as an “applicator”) loaded with an effective HOCl solution needs to be stored within and subsequently dispensed from a container that retains the shelf life of the HOCl solution until the applicator is removed from the container and used for sanitizing and/or disinfecting a hard surface. Previous attempts to provide a suitable substrate material loaded with an effective HOCl solution for sanitizing and disinfecting a hard surface in the form of an applicator have proven unsuccessful, primarily due to the inherent chemistry of HOCl solutions that are not stable over an acceptable shelf-life. A system and method for producing a stable HOCl solution for effective sanitizing and disinfecting using, for example, a conical cell technology is disclosed in U.S. patent application Ser. No. 17/344,951, the entire disclosure of which is incorporated herein by reference.

In view of the foregoing, it is apparent a need exists for improved sanitizing and disinfecting substrates loaded with an effective sanitizing and disinfecting solution. A specific need exists for sanitizing and disinfecting substrates that are loaded with a stable HOCl solution for sanitizing and/or disinfecting a hard surface. Such a substrate would provide a more convenient form of an applicator for effectively sanitizing and disinfecting hard surfaces in institutional facilities, commercial businesses, public areas and private spaces with a stable HOCl solution having an acceptable shelf-life.

Certain aspects, objects, features and advantages of the invention will be made apparent, or will be readily understood and appreciated by those skilled in the relevant art, with reference to the exemplary embodiments of the invention described herein and shown in the accompanying drawing figures. It is intended that the certain aspects, objects, features and advantages of the invention set forth herein be construed in accordance with the ordinary and customary meaning of the elements, terms and limitations of the appended claims given their broadest reasonable interpretation consistent with this written disclosure and the accompanying drawing figures. Some or all aspects, objects, features and advantages of the invention, as well as others not expressly or inherently disclosed herein, may be accomplished by one or more of the exemplary embodiments described herein and shown in the accompanying drawing figures. However, it should be appreciated that the written description and drawing figures are for illustrative purposes only, and that many modifications, substitutions or revisions may be made to the exemplary embodiments without departing from the general concepts of the invention and the intended broad scope and proper construction of the appended claims.

### BRIEF DESCRIPTION OF THE DRAWING FIGURES

The aforementioned aspects, objects, features and advantages of the invention will be more fully understood and appreciated when considered with reference to the accompanying drawing figures, in which like reference characters designate the same or similar parts throughout the several views.

FIG. 1 is a schematic cross-section view of an exemplary embodiment of a sanitizing and disinfecting substrate according to the invention.

FIG. 2 is a perspective view of an exemplary embodiment of a first container for storing and subsequently dispensing sanitizing and disinfecting substrates according to the invention.

FIG. 3 is a perspective view of an exemplary embodiment of a second container for storing and subsequently dispensing sanitizing and disinfecting substrates according to the invention.

FIG. 4 is an exploded perspective view of an exemplary embodiment of a third container for storing and subsequently dispensing sanitizing and disinfecting substrates according to the invention.

### DETAILED DESCRIPTION OF EXEMPLARY EMBODIMENTS

Exemplary embodiments of the present invention are described in greater detail herein and shown in the accompanying drawing figures. The exemplary embodiments described and shown herein are directed to improved sanitizing and disinfecting substrates loaded with an effective sanitizing and disinfecting solution. Advantageous exemplary embodiments described and shown herein are sanitizing and disinfecting substrates loaded with a stable HOCl solution that are stored within and dispensed from a container that maintains an acceptable shelf-life of the HOCl solution. Various aspects, objects, features and advantages of the invention are illustrated by exemplary embodiments of suitable substrates loaded with an effective HOCl solution and disposed within suitable containers for storing and dispensing the substrates. In a particularly advantageous embodiment, a substrate according to the invention comprises a multi-layer, nonwoven fabric material loaded with a stable HOCl solution for effectively sanitizing and/or disinfecting a hard surface, as will be described in greater detail hereafter.

FIG. 1 is a schematic cross-section of an exemplary embodiment of a substrate 10 according to the invention. The substrate 10 is useful, by way of example and not limitation, for effectively sanitizing and/or disinfecting hard surfaces in institutional facilities, commercial businesses, public areas and private spaces with a stable HOCl solution having an acceptable shelf-life. In one aspect, a suitable substrate 10 comprises a nonwoven fabric material. Substrate 10 may be a synthetic nonwoven material comprising one or more layers of plastic fibers, such as high molecular weight polymers like polypropylene (PP), polyethylene (PE), polyester (PET), polyethylene terephthalate (PETE), polyurethane (PUR) and polyamides. Conversely, in another embodiment substrate 10 may be an organic nonwoven material made from cellulosic fibers, such as wood pulp, cotton and linen. As will be readily appreciated by those having ordinary skill in the art, synthetic fabrics are generally more hydrophobic than hydrophilic, as compared with organic fabrics. While being less hydrophilic, synthetic fabrics made of plastic fibers are more resistant to degradation from caustic chemicals, such as chlorine, and in the present case, from the FAC in an HOCl sanitizing and disinfecting solution.

As illustrated in FIG. 1 by way of example and not limitation, the substrate 10 may be a tri-ply laminate synthetic nonwoven fabric comprising a first outer layer 12 of a Spunbond polymer and a second outer layer 14 of a Spunbond polymer, with an inner layer 16 of a Meltblown polymer disposed between the first and second outer layers 12, 14. As such, the synthetic nonwoven fabric substrate 10 depicted in FIG. 1 is typically referred to as Spunbond Meltblown Spunbond (SMS) fabric. In one embodiment, substrate 10 may comprise a first outer (top) layer 12 of Spunbond polypropylene and/or polyester, a second outer (bottom) layer 14 of Spunbond polypropylene and/or polyester, with an inner (middle) layer 16 of Meltblown polypropylene and/or polyester between the first and second outer layers 12, 14. If desired, the outer surfaces of the first and second outer layers 12, 14 may have a soft calendar pattern.

In another embodiment, the substrate 10 may be a synthetic nonwoven fabric formed from a hydroentanglement bonding process by carding, air-laying or wet-laying synthetic fibers. A suitable substrate 10 is a synthetic nonwoven fabric commercially available from Berry Global, Inc. of Evansville, Ind., USA, under the tradename Spinlace®. The Spinlace® nonwoven fabric is formed from Spunbond or SMS synthetic fibers, such as polypropylene (PP) and/or polyester (PET), imaged via a proprietary Apex® technology that is specially designed and formulated for premium applications in the healthcare, hygiene, household cleaning, food service, and industrial wipes markets. By way of example and not limitation, the nonwoven fabric may be a tri-ply laminate comprising first and second outer layers 12, 14 of hydroentangled Spunbond polypropylene (PP) and an inner layer 16 of pulp, such as RISI fluff pulp. In another example, the first and second outer layers 12, 14 may be Spunbond polypropylene (PP) and the inner layer 16 may be polyester (PET). In yet another example, the first and second outer layers 12, 14 may be Spunbond polyester (PET) and the inner layer 16 may be pulp, such as RISI fluff pulp.

In another embodiment, the substrate 10 may be a synthetic nonwoven material formed from one or more layers of polyester (PET) fibers. In general, it is believed that a substrate 10 comprising such a synthetic nonwoven material is particularly well suited for use with HOCl solutions due to the relatively inert nature of the polyester (PET) fibers. In certain embodiments, the synthetic nonwoven material may comprise polyester (PET), wettable PET, or both, with or without a binder system. In one embodiment, the synthetic nonwoven material comprises 50% PET and 50% wettable PET and is commercially known as Spunlace available from Berry Global, Inc. The basis weight may range from about 25 grams per square meter (gsm) to about 80 grams per square meter (gsm) with absorbency capacity of at least 500% and with a hand feel more comparable to a viscose semi-synthetic material than a PET material when wet or otherwise activated. In preferred embodiments, the basis weight of the synthetic nonwoven material is between about 25 gsm and about 50 gsm. In a more preferred embodiment, the basis weight of the synthetic nonwoven material is about 46 gsm. An outer surface of the synthetic nonwoven material may have a soft calendar pattern.

In yet another embodiment, the substrate 10 may be a nonwoven material comprising one or more layers of synthetic fibers and/or organic fibers. As previously mentioned, nonwoven materials made of organic fibers are more hydrophilic than nonwoven materials made of synthetic fibers but are more susceptible to environmental and chemical degradation. Consequently, a substrate 10 formed of a nonwoven material made from synthetic fibers and/or organic fibers, such as cellulosic fibers, may further comprise an optional coating of an additive, such as a surfactant, and/or may be infused with the additive to isolate the organic fibers of the substrate 10 from the caustic FAC present in HOCl solutions. If the substrate 10 is formed as a laminate comprising a plurality of layers of synthetic and/or organic nonwoven material, a suitable water-resistant binding agent or adhesive may be utilized to bond together the individual layers of the substrate 10. Alternatively, the individual layers of the substrate 10 may be bonded together chemically or thermally, for example by ultrasonic welding, or alternatively by hydroentanglement. In any event, the substrate 10 must be constructed in a manner that sufficiently retains (i.e. holds, carries, or the like) the HOCl solution on the substrate 10, while not permitting the FAC present in the HOCl solution to degrade the structural integrity of the substrate 10. By way of example and not limitation, the outer surfaces of the substrate 10 may have a soft calendar pattern or the like believed to provide surface tension for a liquid solution, such as a sanitizing and/or disinfecting solution as described herein.

Regardless, the substrate 10 is loaded with a sanitizing and/or disinfecting solution, indicated generally at 18, and more particularly, a stable HOCl solution. As used herein, the term “loaded” is intended to refer to any suitable means or method for applying the sanitizing and/or disinfecting solution onto the substrate 10. By way of example and not limitation, the sanitizing and/or disinfecting solution 18 may be applied onto the substrate 10 by spraying, coating, dipping, soaking, moistening, pre-moistening or the like. The sanitizing and/or disinfecting solution 18 may be applied onto the substrate 10 during manufacture, or alternatively, may be applied by a user following manufacture and prior to use. As used herein, the term “loaded” is further intended to mean that the substrate 10 is pre-moistened with the HOCl solution 18 during manufacture, or alternatively, the substrate 10 is moistened with the HOCl solution 18 by the end user prior to use. In either case, the substrate 10 is typically moistened up to the saturation point of the nonwoven fabric material such that the substrate 10 retains the HOCL solution 18 on the exposed surfaces of the substrate 10 and thereby acts as an applicator for the HOCL solution 18. By way of example and not limitation, the HOCl solution 18 may be produced by the Electrochemically Activated Water (EAW) system and method utilizing a generator comprising an electrolysis cell as disclosed in the U.S. patent application Ser. No. 17/344,951. However, the HOCl solution 18 may be made by any suitable process that produces a stable sanitizing and disinfecting solution at a desired potential of hydrogen (pH) and concentration of FAC over an acceptable shelf-life.

As will be readily appreciated by those skilled in the art, HOCl solutions 18 used to sanitize and disinfect are driven by an inversely proportional relationship between FAC concentration and dwell time. In other words, as the FAC increases the required dwell time decreases, and vice versa. Therefore, effective and stable concentrations of FAC for sanitizing and disinfecting can reasonably range between about 25 parts per million (ppm) and about 750 parts per million (ppm). Furthermore, different concentrations of FAC can be formulated to produce effective HOCl solutions 18, and thus, substrates loaded with an effective HOCl solution having a desired shelf-life. Consequently, it is desirable to be able to load the substrate 10 with HOCl solutions 18 having a FAC concentration from about 25 ppm to about 750 ppm to provide for various levels of sanitizing and disinfecting. For example, a FAC concentration of from about 250 ppm to about 600 ppm may be preferred for an effective sanitizing and disinfecting HOCl solution 18, while a FAC concentration of from about 400 ppm to about 550 ppm may be more preferred. Likewise, a potential of hydrogen (pH) of from about 4.5 pH to about 7.5 pH may be suitable for an effective HOCl solution 18, while from about 5.0 pH to about 7.0 may be preferred and 5.5 pH may be more preferred.

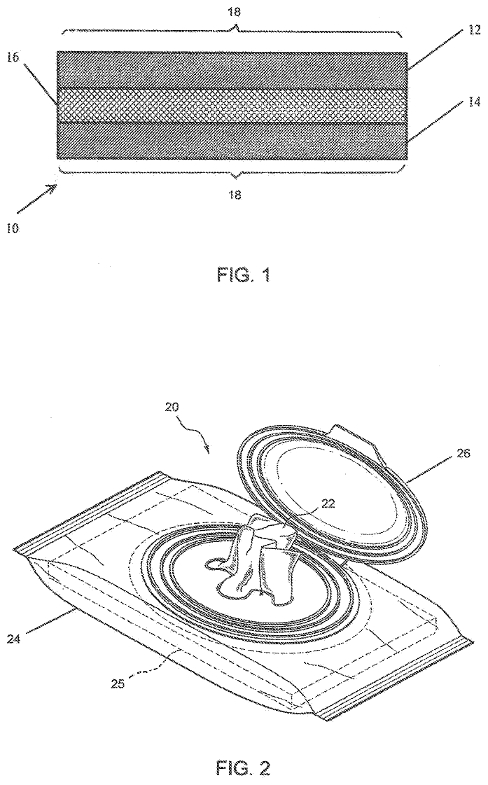

In another aspect, a container is provided for a sanitizing and disinfecting applicator according to the invention. FIG. 2 is a perspective view of an embodiment of a first container 20 for storing and subsequently dispensing sanitizing and disinfecting substrates 10 loaded with an effective HOCl solution 18. In one embodiment, the substrates 10 are loaded with the HOCl solution 18 during manufacture to provide a plurality of pre-moistened HOCl sanitizing and disinfecting applicators like conventional alcohol wipes. Consequently, a container 20 of the type shown and described herein may be referred to as a “wipe flat pack.” Regardless, the sanitizing and disinfecting applicators or wipes, indicated generally by reference character 22 in FIG. 2, are formed from the substrate 10 and the HOCl solution 18 described herein. In the embodiment shown in FIG. 2, a plurality of sheets of the pre-moistened HOCl wipes 22 are disposed within the container 20 such that individual wipes 22 may be dispensed from the container 20. The container 20 may comprise a pack or similar housing, receptacle or the like, referred to herein as a pouch 24, defining a generally hollow interior 25 for receiving the pre-moistened HOCl wipes 22. Pouch 24 may be made of an opaque plastic material and/or may be provided with an inner lining, such as a foil film, to prevent transmission of ultraviolet light onto the HOCl wipes 22 prior to use. Pouch 24 is sealed during manufacture with a removable and preferably resealable cover 26 that provides access to the HOCl wipes 22 disposed within the interior 25 of the container 20. The individual sheets of wipes 22 may be separated by perforations or may be folded and stacked in a known manner to facilitate removal of each wipe 22 individually from the pouch 24. As a result, the container 20 provides a reusable package of pre-moistened (i.e. wetted) HOCl wipes 22 in a convenient form available to be used for sanitizing and disinfecting hard surfaces in institutional facilities, commercial businesses, public areas and private spaces.

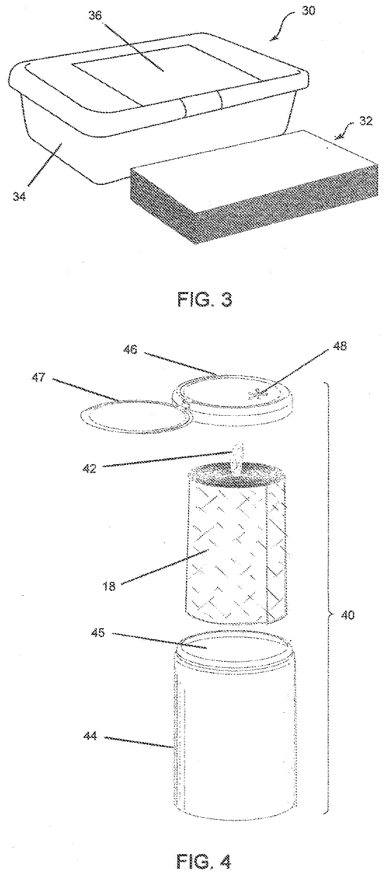

FIG. 3 is a perspective view of an embodiment of a second container 30 according to the invention for storing and subsequently dispensing sanitizing and disinfecting substrates 10 loaded with an effective HOCl solution 18 according to the invention. In one embodiment, the substrates 10 are loaded with the HOCl solution 18 during manufacture to provide a plurality of pre-moistened HOCl sanitizing and disinfecting applicators like conventional alcohol wipes. Consequently, a container 30 of the type shown and described herein may be referred to as a “wipe box.” In another embodiment, the substrates 10 are disposed within the container 30 and the HOCl solution 18 is subsequently introduced into the container 30 such that the substrates 10 are wetted with the HOCl solution 18, preferably to the point of saturation. Regardless, the sanitizing and disinfecting applicators or wipes, indicated generally by reference character 32 in FIG. 3, are formed from the substrate 10 and the HOCl solution 18 described herein. In the embodiment shown in FIG. 3, a plurality of sheets of the pre-moistened HOCl wipes 32 are disposed within the container 30 such that individual wipes 32 may be dispensed from the container 30. The container 30 may comprise a housing, receptacle or the like, referred to herein as a box 34, defining a generally hollow interior for receiving the pre-moistened HOCl wipes 32. Box 34 may be made of an opaque plastic material and/or may be provided with an inner lining, such as a foil film, to prevent transmission of ultraviolet light onto the HOCl wipes 32 prior to use. Box 34 is sealed during manufacture with an openable and preferably resealable, for example a hinged, cover 36 that provides access to the HOCl wipes 32 disposed within the container 30. The individual sheets of wipes 32 may be folded and stacked in a known manner to facilitate removal of each wipe 32 individually from the box 34. As a result, container 30 provides a reusable package of pre-moistened (i.e. wetted) HOCl wipes 32 in a convenient form available to be used for sanitizing and disinfecting hard surfaces in institutional facilities, commercial businesses, public areas and private spaces.

FIG. 4 is a perspective view of an embodiment of a third container 40 according to the invention for storing and subsequently dispensing sanitizing and disinfecting substrates 10 loaded with an effective HOCl solution 18 according to the invention. In one embodiment, the substrates 10 are loaded with the HOCl solution 18 during manufacture to form a wet roll of pre-moistened HOCl sanitizing and disinfecting applicators. In another embodiment, substrates 10 similar to a dry roll of cloth or paper towels are disposed within the container 40 and the HOCl solution 18 is introduced into the container 40 such that the substrates 10 are wetted with the HOCl solution 18, preferably to the point of saturation. The HOCl solution 18 may be generated and introduced into the container 40 with a roll of the substrates 10 at the time of manufacture. Alternatively, the container 40 is manufactured and provided to the user with a dry roll of the substrates 10 disposed within the container 40. In this manner, the user can introduce the HOCl solution 18 into the container 40 as needed to maximize the shelf-life of the sanitizing and disinfecting solution. The HOCl solution 18 may be pre-packaged in a hermetically sealed dispenser and stored until needed. Alternatively, the HOCl solution 18 may be generated on-site using HOCl generation equipment, for example of the type disclosed in the U.S. patent application Ser. No. 17/344,951 and introduced into the container 40 at the time of use. Regardless, the sanitizing and disinfecting applicators indicated generally by reference character 42 in FIG. 4 are formed from the substrate 10 and the HOCl solution 18 described herein.

In the embodiment shown in FIG. 4, the substrates 10 (i.e. the applicators 42) are produced in a continuous sheet form and converted to a perforated roll form. In one embodiment, the perforated roll, commonly referred to as a “log”, comprises a plurality of individual polyester microfiber dry wipes having high surface tension with a length (or width) dimension of at least about six inches and a height dimension of at least about nine inches. Container 40 may comprise a housing, receptacle or the like, referred to herein as a canister 44, defining a generally hollow interior 45 for receiving the perforated roll of dry applicators 42. The canister 44 may be made of an opaque plastic material and/or may be provided with an inner lining, such as a foil film, to prevent transmission of ultraviolet light onto the wipes 42 prior to use. Canister 44 is closed during manufacture with a removable and/or openable cover 46 that provides access to the interior 45 of the canister 44 and to the applicators 42. As illustrated herein, canister 44 and cover 46 may be removably engaged and disengaged by external and internal threads, respectively. In this manner, cover 46 may be removed from canister 44 so that the interior 45 of the canister 44 can be filled with the HOCl solution 18 to wet the perforated roll of dry substrates 10 with the HOCl solution 18.

As previously described, the substrates 10 and HOCl solution 18 may be generated at an off-site location and provided separately or together with or without container 40. Alternatively, the HOCl solution 18 may be generated off-site and introduced into the container 40 with the perforated roll of dry substrates 10 either at the time of manufacture or on-site prior to use. Alternatively, the HOCl solution 18 may be generated on-site and introduced into the container 40 with the perforated roll of dry substrates 18 at the time of use. Still further, the HOCl solution 18 may be provided in a spray cart, spray bottle or liquid dispenser and sprayed or poured onto a dry applicator 42 dispensed from the canister 44 of the container 40. Cover 46 is preferably provided with a hinged portion 47 and/or a slotted opening 48 for readily dispensing a perforated sheet of wet or dry applicator 42 without removing the cover 46 from the canister 44. As a result, container 40 provides a reusable package of pre-moistened (i.e. wetted) or dry applicators 42 in a convenient form available to be used for sanitizing and disinfecting hard surfaces in institutional facilities, commercial businesses, public areas and private spaces.

The substrate 10 loaded with sanitizing and disinfecting HOCl solution 18 may be provided in the form of a single layer or multi-layer wet wipe, wet hand sanitizing wipe, wet mitt, wet cleaning cloth, or wet cleaning pad for use with or without a cleaning, sanitizing or disinfecting implement, such as a wand, brush, mop, mop head or the like. As used herein, the term “wet” is intended to mean that the substrate 10 is pre-moistened or moistened in situ with the HOCl solution 18, preferably up to the point of the substrate 10 being saturated with the HOCl solution 18. Alternatively, the substrate 10 may be provided in the form of a single layer or multi-layer dry wipe, dry mitt, dry cleaning cloth or dry cleaning pad for use with or without a cleaning, sanitizing or disinfecting implement, such as a wand, brush, mop, mop head or the like, that is configured to be subsequently moistened with a HOCl solution 18. In the latter instance, the HOCl solution 18 may be generated off-site and delivered with or without the substrate 10, or alternatively, generated on-site at a location where the substrate 10 is to be moistened prior to use so as to maximize the shelf-life of the sanitizing and disinfecting HOCl solution 18. In any of the previous examples, the substrate 10 may be generically referred to as a “wipe” or as an “applicator,” subject to any regulatory requirement for approval and/or authorization.

The foregoing detailed description of exemplary embodiments of improved substrates loaded with sanitizing and disinfecting solutions is merely illustrative of the general concepts and principles of the present invention. Regardless of the foregoing detailed description and illustrated embodiments, various other configurations of the substrates and solutions, as well as reasonable equivalents thereof, will be readily apparent and understood by those having ordinary skill in the art. Accordingly, equivalents to the embodiments described in the written description and shown in the accompanying drawing figures are intended to be encompassed by the broadest reasonable interpretation and construction of the appended claims. Furthermore, as numerous modifications and changes to the exemplary embodiments will readily occur to those skilled in the art, the present invention is not to be limited to the specific configuration, construction, materials, manner of use and operation shown and described herein. Instead, all reasonably predictable and suitable equivalents and obvious modifications to the invention should be determined to fall within the scope of the appended claims given their broadest reasonable interpretation and construction in view of the accompanying written description and drawing figures in view of the combined teachings of the disclosures of the relevant prior art.

That which is claimed is:

1. A sanitizing and disinfecting substrate comprising:
a nonwoven material; and
a hypochlorous acid (HOCl) solution having a free available chlorine (FAC) concentration of between about 25 ppm and about 750 ppm.

2. The sanitizing and disinfecting substrate of claim 1, wherein the nonwoven fabric material is a synthetic nonwoven fabric material comprising plastic fibers.

3. The sanitizing and disinfecting substrate of claim 2, wherein the synthetic nonwoven fabric material comprises a laminate having a plurality of layers.

4. The sanitizing and disinfecting substrate of claim 3, wherein the laminate comprises a first outer layer of a Spunbond polymer and a second outer layer of a Spunbond polymer.

5. The sanitizing and disinfecting substrate of claim 4, wherein at least one of the first outer layer of the Spunbond polymer and the second outer layer of the Spunbond polymer is selected from the group consisting of polypropylene (PP) and polyester (PET).

6. The sanitizing and disinfecting substrate of claim 5 wherein the laminate further comprises an inner layer disposed between the first outer layer and the second outer layer, and wherein the inner layer comprises at least one of polyester (PET) and pulp.

7. The sanitizing and disinfecting substrate of claim 3, wherein the laminate of the nonwoven fabric material is a Spunbond Meltblown Spunbond (SMS) tri-ply laminate and at least one of the first and second outer layers of the laminate has a soft calendar pattern.

8. The sanitizing and disinfecting substrate of claim 3, wherein the layers of the laminate are bonded together by at least one of adhesive bonding, chemical bonding and thermal bonding.

9. The sanitizing and disinfecting substrate of claim 1, wherein the nonwoven fabric material is an organic nonwoven fabric material comprising cellulosic fibers.

10. The sanitizing and disinfecting substrate of claim 9, wherein the organic nonwoven fabric material has a coating comprising a synthetic material.

11. A combination substrate and container for providing a sanitizing and disinfecting applicator, comprising:
at least one substrate formed from a nonwoven fabric material that is loaded with a hypochlorous acid (HOCl) solution; and
a container configured for storing and subsequently dispensing the at least one substrate.

12. The combination substrate and container of claim 11, wherein the at least one substrate is pre-moistened with the HOCl solution and disposed within the container during manufacture.

13. The combination substrate and container of claim 11, wherein the at least one substrate is disposed within the container during manufacture and the HOCl solution is subsequently introduced into the container to moisten the at least one substrate with the HOCl solution prior to use.

14. The combination substrate and container of claim 11, wherein the at least one substrate comprises a plurality of substrates that are pre-moistened and pre-packaged within the container.

15. The combination substrate and container of claim 14, wherein the plurality of substrates is provided in the form of individual sheets that are folded and stacked within the container to be individually dispensed from the container.

16. The combination substrate and container of claim 11, wherein the at least one substrate comprises a plurality of substrates provided in the form of a perforated roll and configured to be disposed within the container.

17. The combination substrate and container of claim 16, wherein the container is subsequently filled with the HOCl solution to moisten the plurality of substrates prior to use.

18. The combination substrate and container of claim 17, wherein the HOCl solution is generated on-site and the container is filled with the HOCl solution prior to use.

19. The combination substrate and container of claim 11, wherein the at least one substrate is subsequently dispensed from the container and moistened with the HOCl solution at the time of use.

20. The combination substrate and container of claim 11, wherein the HOCl solution has a free available chlorine (FAC) concentration of from about 25 ppm to about 750 ppm and a potential of hydrogen (pH) of from about 4.5 pH to about 7.5 pH.
